(19) United States
(12) Patent Application Publication
(10) Pub. No.: US 2023/0341229 A1
(43) Pub. Date: Oct. 26, 2023

(54) VEHICLE-BASED MULTI-MODAL TRIP PLANNING AND EVENT COORDINATION

(71) Applicant: Ford Global Technologies, LLC, Dearborn, MI (US)

(72) Inventors: Stuart C. Salter, White Lake, MI (US); Brendan Diamond, Grosse Pointe, MI (US); David Kennedy, Canton, MI (US); Annette Huebner, Highland, MI (US); Lucretia Williams, Bloomfield Hills, MI (US); Monica Lam, Dearborn, MI (US)

(73) Assignee: Ford Global Technologies, LLC, Dearborn, MI (US)

(21) Appl. No.: 17/724,707

(22) Filed: Apr. 20, 2022

(51) Int. Cl. G01C 21/34; G01C 21/36; G06V 40/10; G06V 20/56

(52) U.S. Cl. CPC G01C 21/3423; G01C 21/3602; G01C 21/3679; G01C 21/3661; G06V 40/103; G06V 20/56; G06Q 30/0633

(57) ABSTRACT

This disclosure describes systems, methods, and devices related to multi-modal trip planning and event coordination. A method may include detecting, by a first device, based on first location data of a vehicle, that the vehicle has arrived at a destination location for a passenger of the vehicle; generating directions from the vehicle at the destination location to a physical structure at the destination location; presenting the directions; detecting an image of the passenger exterior to the vehicle at the destination location; identifying, by the at least one processor, based on the image, user information associated with the passenger at the destination location; detecting, based on the user information and second location data associated with a second device of the passenger, a disorientation event associated with the passenger; generating, based on the detecting of the disorientation event, instructions to be presented to the passenger; and presenting the instructions.

### TECHNICAL FIELD

This disclosure generally relates to systems and methods for assisting people with cognitive disabilities and, more particularly, to vehicle-based multi-modal trip planning for people with cognitive disabilities.

### BACKGROUND

Autonomous vehicles increasingly are being used. Some passengers of autonomous vehicles may experience cognitive disabilities than disorient them inside and outside of a vehicle. An autonomous vehicle may drive a passenger to a location, but the passenger may experience disorientation even after exiting the vehicle.

### BRIEF DESCRIPTION OF THE DRAWINGS

The present description will be better understood from the following detailed description read in light of the accompanying drawings, wherein like reference numerals are used to designate like parts in the accompanying description.

FIG. 1 shows an example process for multi-modal trip planning and event coordination in accordance with one or more embodiments of the disclosure.

FIG. 2 shows an example system for multi-modal trip planning and event coordination in accordance with one or more embodiments of the disclosure.

FIG. 3 shows an example in-vehicle system for multi-modal trip planning and event coordination in accordance with one or more embodiments of the disclosure.

FIG. 4 illustrates a flow diagram of an illustrative process for multi-modal trip planning and event coordination in accordance with one or more embodiments of the disclosure.

FIG. 5 is a block diagram illustrating an example of a computing device or computer system upon which any of one or more techniques (e.g., methods) may be performed in accordance with one or more embodiments of this disclosure.

### DETAILED DESCRIPTION

### Overview

Passengers of vehicles may benefit from being driven to a destination location, but may experience disorientation once they exit the vehicle, even at the destination location. For example, a passenger may be driven to a grocery store in an autonomous vehicle, but after arriving at the grocery store, may forget why they are there, where to go, what to do, and/or how to return to the vehicle. Alternatively, a vehicle driver may become lost while driving a vehicle.

Embodiments described herein detect when a vehicle passenger experiences a disorientation event when outside of a vehicle, presenting to the passenger and/or others (e.g., family, friends, medical professionals, and the like) instructions regarding where the passenger is, where the passenger is supposed to go, what tasks the passenger is to complete, what timeframe the passenger is supposed to be at a destination, how to get from the vehicle to a physical location (e.g., a physical structure such as a building for a store, office, doctor's office, residence, etc.), how to navigate within a physical structure (e.g., directions inside of a building), and/or how to return to the vehicle from the physical structure. The instructions also may include updates to another party (e.g., family, friends, medical professionals, and the like)

In some embodiments, a disorientation event of a person may include memory loss, inability to navigate to a location, being at a location for longer than an expected time, being stationary or within a small area for longer than a threshold time, moving at a speed lower than a threshold, being outside of a specified location/boundary (e.g., based on geographical coordinates, geo-fencing, etc.), having vital signs that are above or below respective thresholds (e.g., indicative of fatigue, stress, etc.), taking too long to complete a task or set of tasks, and the like.

In some embodiments, detection of a disorientation event may use a combination of image data, device location data, device motion data, and/or biometric sensor data. For example, with user consent and in accordance with relevant laws, a user's location may be monitored using device location data (e.g., global navigation satellite system data, Wi-Fi data, Bluetooth data, etc.), a user's state of being may be monitored using images and/or sensor data (e.g., images used for analysis to detect facial expressions, items in a person's possession, attire, gait, injuries, and the like, and biometric sensor data such as body temperature, heartrate, breathing rate, pulse, etc.), and a user's activity may be monitored using device motion data (e.g., accelerometer data indicative of a person falling down or moving in a manner that is unexpected or indicative of stress). A system associated with a vehicle may detect disorientation events.

In some embodiments, based on the detection of a disorientation event, a system associated with a vehicle may generate instructions such as maps, directions to and from multiple locations, lists of tasks to complete and/or items to purchase or possess, expected time durations for a person to be at respective locations and/or whether those time durations have expired or allow for more time at a location, and user queries to confirm (e.g., with a return response or absence of a return response) whether a user is okay and/or has any queries regarding location, tasks, time duration, etc. The instructions also may be provided to other parties to inform them of a passenger's status and/or the maps, directions to and from multiple locations, lists of tasks to complete and/or items to purchase or possess, expected time durations for a person to be at respective locations and/or whether those time durations have expired or allow for more time at a location, and user queries to confirm whether a user is okay and/or has any queries regarding location, tasks, time duration, etc. The instructions may include any combination of audio and/or visual data (e.g., audio and/or video instructions, etc.).

In some embodiments, the instructions may be generated based on factors such as task completion rate (e.g., historical data indicative of how often a person performs a task or arrives at a location within an amount of time), the type and/or severity of a disorientation (e.g., a cardiac event indicated by biometric sensor data may require sending an emergency medical team to a person, whereas a person who may need help remembering an event or directions to a location may need a reminder, map, directions, etc.). The instructions may break up trips (e.g., from one destination to at least one other destination) by adding rest time (e.g., elongating time periods for tasks/locations), additional tasks (e.g., food or bathroom breaks, etc.), adding destinations (e.g., for breaks, meals, etc.), or by reducing destinations and/or tasks.

In some embodiments, the instructions and/or criteria used to detect a disorientation event may vary based on factors such as a time of day, the disorientation condition (e.g., profiles based on a condition such a Alzheimer's or Autism and including preset and/or adjusted criteria, such as thresholds for respective types of data), and/or environmental conditions (e.g., lighting, temperature, crowded or sparse areas, etc.). For example, at night, disorientation may be more severe, and disorientation may be more likely in crowded areas or certain types of venues (e.g., a grocery or department store) than other venues (e.g., based on venue type or size). In this manner, time of day and/or environmental conditions may alter threshold times, geo-fencing, and the like, with which to detect disorientation events, and also may alter instructions (e.g., directions to and from locations may avoid certain areas that are crowded, darker, or the like, in favor of less crowded areas, areas with better lighting, etc.).

In some embodiments, the system associated with a vehicle may detect a root cause of a detected disorientation event (e.g., to update a profile to use in detecting a disorientation event and/or to generate instructions in response to a disorientation event). The system may identify conditions that may have caused the disorientation event, such as location, time of day, biometric data, health conditions, environmental conditions, length of time to complete a task, and the like. The instructions generated by the system may indicate the root cause conditions to the appropriate parties. The system also may avoid the root cause conditions in future situations. For example, the system may set threshold amounts of time to complete tasks to reduce the risk of a person becoming disoriented during a time that is too long, may avoid generating directions using certain locations and/or environments (e.g., low-lighting environments, crowded environments, etc.), and the like. When instructions for a user require the root cause conditions (e.g., a longer trip, a crowded location, etc.), the system may generate instructions indicating that the user should travel with a companion to avoid or assist with a disorientation event.

In some embodiments, the vehicle may not be autonomous, and the person being monitored for a disorientation event may be the driver. The image data, location data, and/or sensor data of the driver may be used similarly to detect whether the driver is confused, not using an expected route or not within a threshold distance of an expected distance given an expected route, whether biometric data indicate that the person is having an event (e.g., cardiac arrest, fatigue, etc.). The instructions may be generated and presented using an in-vehicle system while the person is driving.

In some embodiments, the system associated with a vehicle may include processing, communication, and sensor devices for detecting disorientation events, receiving user inputs, generating maps and directions, presenting maps and directions, generating and presenting instructions, and sending instructions to be presented by one or more devices. For example, the vehicle's hardware and software may perform the detection, processing, sending, and presentation of data, and/or a remote system (e.g., a sever-based system) may be in communication with the vehicle to receive data from the vehicle and/or user devices, to analyze the data to detect events, and to generate instructions to be sent to the vehicle and/or other devices for presentation.

### Embodiments

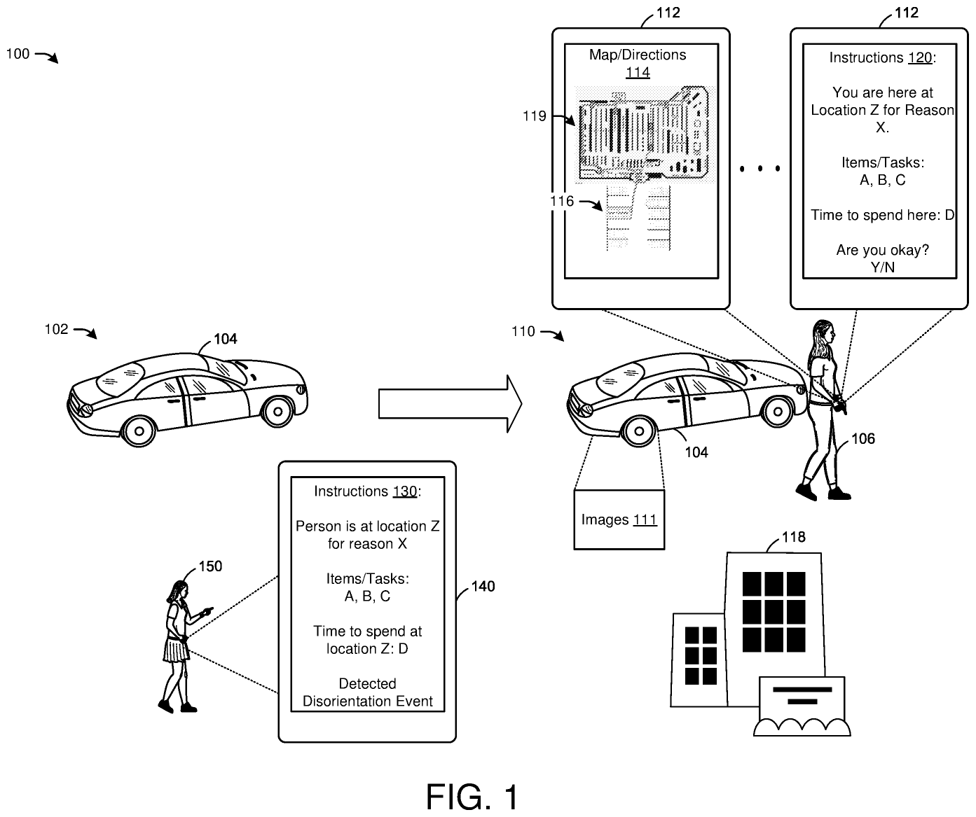

FIG. 1 shows an example process 100 for multi-modal trip planning and event coordination in accordance with one or more embodiments of the disclosure.

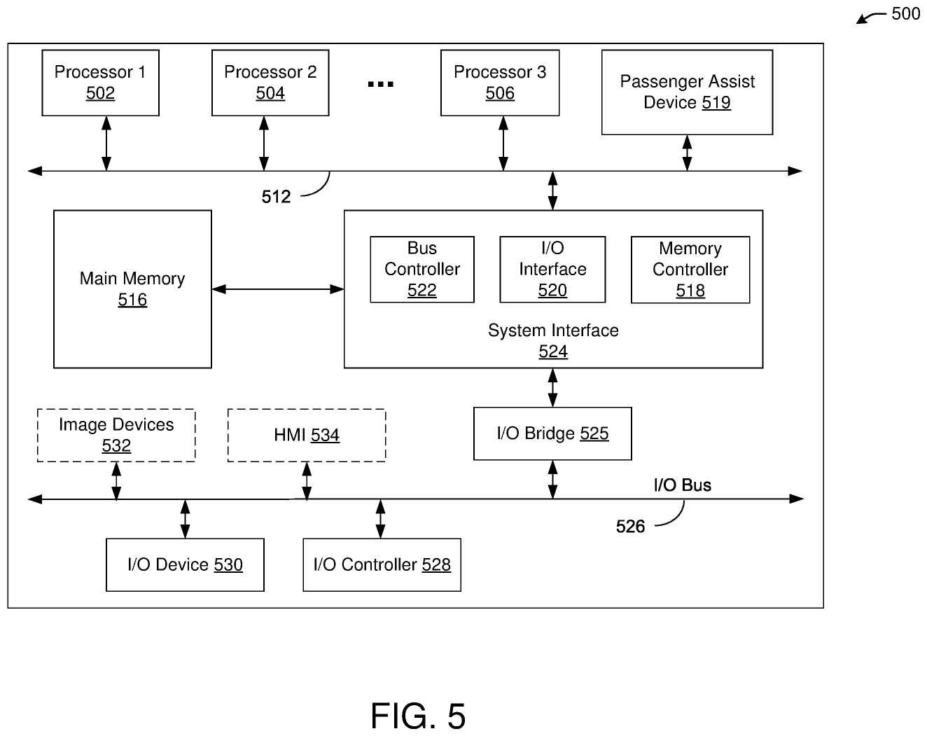

Referring to FIG. 1, the process 100 includes step 102 (e.g., at a first time) when a vehicle 104 (e.g., an autonomous vehicle or non-autonomous vehicle) may be driving from one location to another location (e.g., a destination location based on a user input). A passenger 106 may be a passenger of the vehicle 104. At step 110, the vehicle 104 may arrive at a destination location, and the passenger 106 may exit the vehicle 104. Once the passenger 106 is outside of the vehicle 104, one or more cameras of the vehicle (e.g., see FIG. 5) may capture one or more images 111 of the passenger 106 to use for analysis in detecting a disorientation event of the passenger 106 (e.g., with user consent and in accordance with relevant laws).

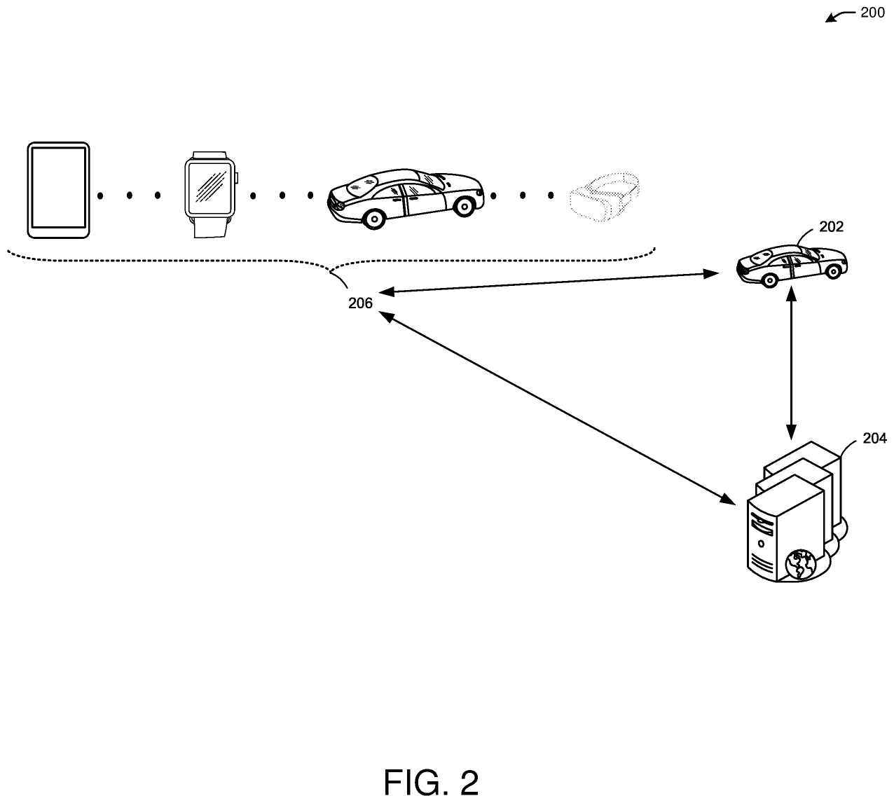

Still referring to FIG. 1, based on detection of a disorientation event, a system of the vehicle 104 or in communication with the vehicle 104 (e.g., see FIG. 2) may generate and send information to a device 112 of the passenger 106, such as maps/directions 114 (e.g., from a location 116 of the vehicle 104 at the destination location, such as where the passenger 106 exits the vehicle 104, to a physical location 118, such as a building or residence, and/or to one or more locations 119 inside of the physical location, such as locations of items to be purchased, offices, or the like. The device 112 also may receive instructions 120 generated and sent by the system of the vehicle 104 or in communication with the vehicle 104. For example, the instructions 120 may include an indication of the physical location 118 and/or why the passenger 106 is there (e.g., a store, a residence, a doctor's office, etc., for shopping, a schedule appointment, etc.). The reason why the passenger 106 is at the physical location 118 may be provided by a user input from the passenger 106 or another user, or may be learned (e.g., from a calendar event and related data, such as provided by the device 112). The instructions 120 also may include items or tasks for the passenger 106 at the physical location 118, such as a shopping list, a to-do list, etc., and may be provided by a user input from the passenger 106 or another user, or may be learned (e.g., from a calendar event and related data, shopping lists, etc. such as provided by the device 112). The instructions 120 also may provide expected time durations for the passenger 106 to spend at the physical location 118 and/or any locations 119 within the physical location 118.

Still referring to FIG. 1, with user consent and in accordance with relevant laws, the system of the vehicle 104 or in communication with the vehicle 104 may generate and send instructions 130 for presentation at a device 140 of another user 150 to inform the user 150 that the passenger 106 is at the physical location 118, for a particular reason, tasks/items for the passenger 106 to complete/purchase at the physical location 118, time to spend at the physical location 118 and/or any locations 119 within the physical location, and any indication of a detected disorientation event of the passenger 106.

In some embodiments, a disorientation event of the passenger 106 may include memory loss, inability to navigate to a location (e.g., the physical location 118 and/or any of the locations 119), being at a location (e.g., the physical location 118 and/or any of the locations 119) for longer than a threshold time, being stationary or within a small area for longer than a threshold time (e.g., based on location data of the device 112 and/or device motion data, such as accelerometer data, of the device 112), moving at a speed lower than a threshold, being outside of a specified location/boundary (e.g., based on geographical coordinates of the device 112, geo-fencing, etc.), having vital signs that are above or below respective thresholds (e.g., indicative of fatigue, stress, etc. as indicated by sensor data of the device 112 or other devices, such as shown in FIG. 2), taking too long to complete a task or set of tasks, and the like.

In some embodiments, detection of a disorientation event may use a combination of image data (e.g., from the images 111), device location data, device motion data, and/or biometric sensor data. For example, with user consent and in accordance with relevant laws, the passenger's location may be monitored using device location data (e.g., global navigation satellite system data, Wi-Fi data, Bluetooth data, etc.), a user's state of being may be monitored using the images 111 and/or sensor data (e.g., images used for analysis to detect facial expressions, items in the passenger's possession, attire, gait, injuries, and the like, and biometric sensor data such as body temperature, heartrate, breathing rate, pulse, etc.), and the passenger's activity may be monitored using device motion data (e.g., accelerometer data indicative of the passenger 106 falling down or moving in a manner that is unexpected or indicative of stress).

In some embodiments, based on the detection of a disorientation event, the system associated with the vehicle 104 may generate instructions such as maps, directions to and from multiple locations, lists of tasks to complete and/or items to purchase or possess, expected time durations for a person to be at respective locations and/or whether those time durations have expired or allow for more time at a location, and user queries to confirm (e.g., with a return response or absence of a return response) whether a user is okay and/or has any queries regarding location, tasks, time duration, etc. The instructions also may be provided to other parties to inform them of the passenger's status and/or the maps, directions to and from multiple locations, lists of tasks to complete and/or items to purchase or possess, expected time durations for the passenger to be at respective locations and/or whether those time durations have expired or allow for more time at a location, and user queries to confirm whether the passenger is okay and/or has any queries regarding location, tasks, time duration, etc. The instructions may include any combination of audio and/or visual data (e.g., audio and/or video instructions, etc.).

In some embodiments, the instructions may be generated based on factors such as task completion rate (e.g., historical data indicative of how often a person, such as the passenger 106 or another person or group of persons, performs a task or arrives at a location within an amount of time), the type and/or severity of a disorientation (e.g., a cardiac event indicated by biometric sensor data may require sending an emergency medical team to a person, whereas a person who may need help remembering an event or directions to a location may need a reminder, map, directions, etc.). The instructions may break up trips (e.g., from one destination to at least one other destination) by adding rest time (e.g., elongating time periods for tasks/locations), additional tasks (e.g., food or bathroom breaks, etc.), adding destinations (e.g., for breaks, meals, etc.), or by reducing destinations and/or tasks.

In some embodiments, the instructions and/or criteria used to detect a disorientation event may vary based on factors such as a time of day and/or environmental conditions (e.g., lighting, temperature, crowded or sparse areas, etc.). For example, at night, disorientation may be more severe, and disorientation may be more likely in crowded areas or certain types of venues (e.g., a grocery or department store) than other venues (e.g., based on venue type or size). In this manner, time of day and/or environmental conditions may alter threshold times, geo-fencing, and the like, with which to detect disorientation events, and also may alter instructions (e.g., directions to and from locations may avoid certain areas that are crowded, darker, or the like, in favor of less crowded areas, areas with better lighting, etc.).

FIG. 2 shows an example system 200 for multi-modal trip planning and event coordination in accordance with one or more embodiments of the disclosure.

Referring to FIG. 2, the system 200 may include a vehicle 202 (e.g., representing the vehicle 104 of FIG. 1) in communication with a remote system 204 (e.g., a server-based system). The vehicle 202 and/or the remote system 204 may be in communication with one or more devices 206 (e.g., representing the device 112 of FIG. 1, the device 140 of FIG. 1, and/or other devices of one or more users, such as smartphones, tablets, wearable devices, vehicle devices, augmented reality devices, virtual reality devices, and the like). Using data of the one or more devices 206 and the vehicle 202, the vehicle 202 and/or the remote system 204 may detect disorientation events, generate maps and directions for presentation (e.g., the map/directions 114 of FIG. 1), generate instructions for presentation (e.g., the instructions 120 and 130 of FIG. 1), and analyze images (e.g., the images 111 of FIG. 1) for user information to consider when detecting disorientation events.

In some embodiments, the system associated with an autonomous vehicle may include processing, communication, and sensor devices for detecting disorientation events, receiving user inputs, generating maps and directions, presenting maps and directions, generating and presenting instructions, and sending instructions to be presented by one or more devices. For example, the vehicle's hardware and software may perform the detection, processing, sending, and presentation of data, and/or the remote system 204 may be in communication with the vehicle 202 to receive data from the vehicle 202 and/or the one or more devices 206, to analyze the data to detect events, and to generate instructions to be sent to the vehicle and/or other devices for presentation. The vehicle 202 and/or the remote system 204 may include components shown in FIG. 5. The one or more devices 206 may include components shown in FIG. 5, may include one or more biometric sensors (not shown) for detecting biometric data to send to the vehicle 202 and/or the remote system 204, and may include one or more device motion sensors (e.g., accelerometers, not shown) for detecting device motion data to send to the vehicle 202 and/or the remote system 204.

In one or more embodiments, the vehicle 202, the remote system 204, and/or the one or more devices 206 may include a personal computer (PC), a wearable wireless device (e.g., bracelet, watch, glasses, ring, etc.), a desktop computer, a mobile computer, a laptop computer, an Ultrabook™ computer, a notebook computer, a tablet computer, a server computer, a handheld computer, a handheld device, an internet of things (IoT) device, a sensor device, a PDA device, a handheld PDA device, an on-board device, an off-board device, a hybrid device (e.g., combining cellular phone functionalities with PDA device functionalities), a consumer device, a vehicular device, a non-vehicular device, a mobile or portable device, a non-mobile or non-portable device, a mobile phone, a cellular telephone, a PCS device, a PDA device which incorporates a wireless communication device, a mobile or portable GPS device, a DVB device, a relatively small computing device, a non-desktop computer, a video device, an audio device, an A/V device, a set-top-box (STB), a Blu-ray disc (BD) player, a BD recorder, a digital video disc (DVD) player, a high definition (HD) DVD player, a DVD recorder, a HD DVD recorder, a personal video recorder (PVR), a broadcast HD receiver, a video source, an audio source, a video sink, an audio sink, a stereo tuner, a broadcast radio receiver, a flat panel display, a personal media player (PMP), a digital video camera (DVC), a digital audio player, a speaker, an audio receiver, an audio amplifier, a gaming device, a data source, a data sink, a digital still camera (DSC), a media player, a smartphone, a television, a music player, or the like.

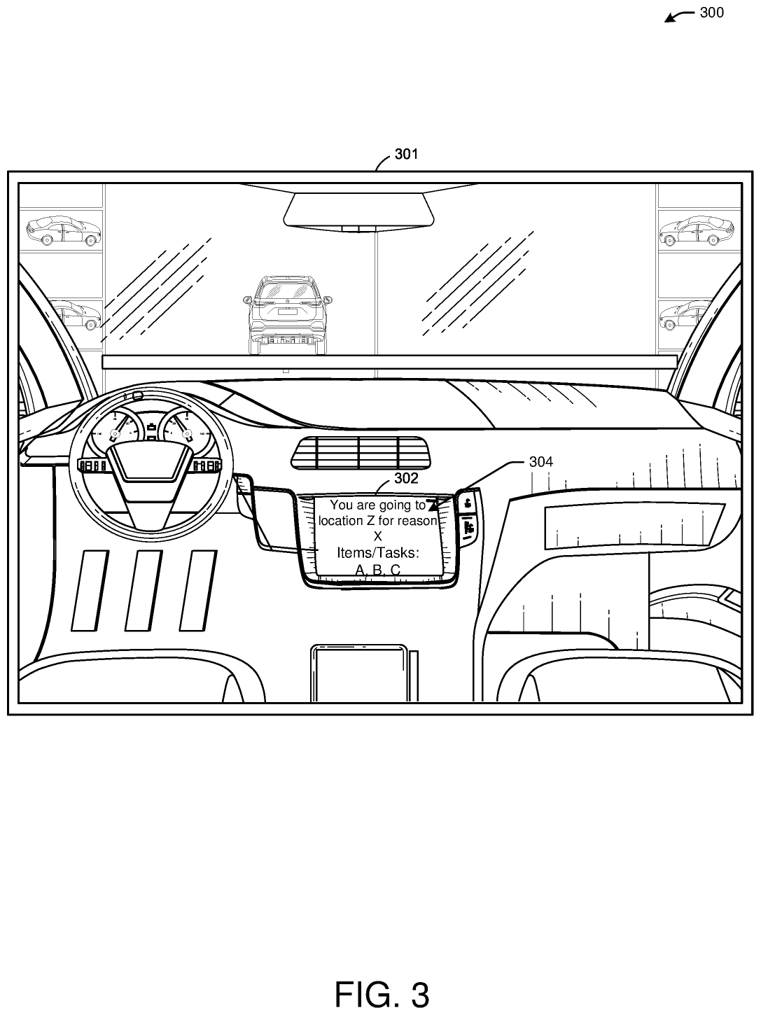

FIG. 3 shows an example in-vehicle system 300 for multi-modal trip planning and event coordination in accordance with one or more embodiments of the disclosure.

Referring to FIG. 3, the in-vehicle system 300 may represent the interior of a vehicle 301 (e.g., the vehicle 104 of FIG. 1, the vehicle 202 of FIG. 2). The in-vehicle system 300 may include an infotainment system 302, such as a human machine interface (HMI) of the vehicle, which may present instructions 304 (e.g., to the passenger 106 of FIG. 1). The instructions 304 may indicate one or more destination locations to where the passenger is being taken by the vehicle, reasons for being taken to the location (e.g., tasks to complete, items to purchase, appointments, etc.), time durations for the passenger to spend at a particular location, and the like (e.g., similar to the instructions 120 of FIG. 1). In this manner, the instructions 304 may be presented to the passenger using vehicle presentation, including before the passenger exits the vehicle (e.g., to remind the passenger of where they are going and what they are expected to do for a particular amount of time). Once the passenger exits the vehicle 301, however, the process 100 of FIG. 1 may execute.

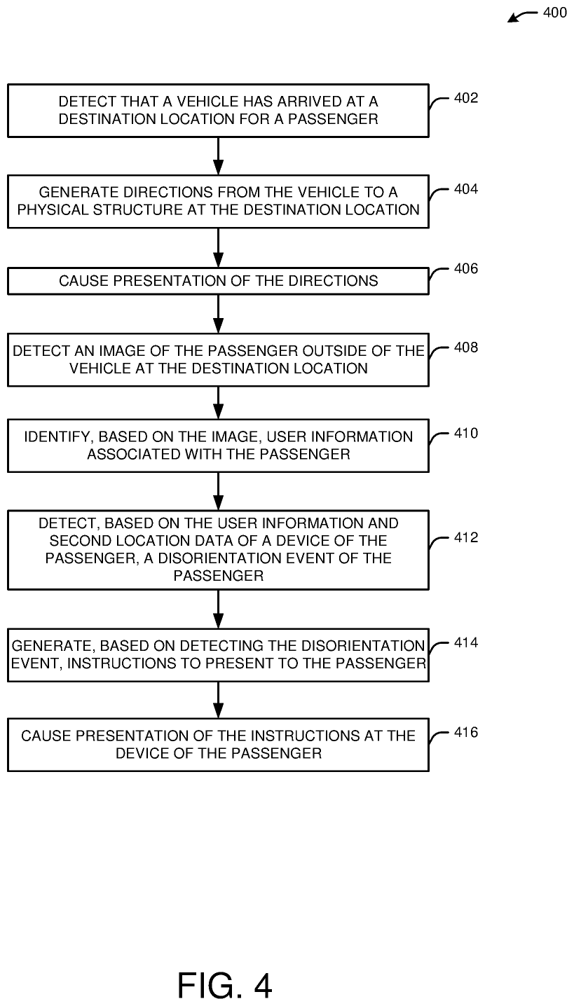

FIG. 4 illustrates a flow diagram of an illustrative process 400 for multi-modal trip planning and event coordination in accordance with one or more embodiments of the disclosure.

At block 402, a device (e.g., the passenger assist device 519 of FIG. 5, implemented as part of the vehicle 202 and/or the remote system 204 of FIG. 2) may detect that a vehicle (e.g., the vehicle 104 of FIG. 1, the vehicle 202, the vehicle 301 of FIG. 3) has arrived at a destination location. The detection may be based on location data of the vehicle transporting the passenger and/or location data of a device of the passenger. The destination location may be identified based on a user input from the passenger, or learned (e.g., from a scheduled event on the device or another device). The device may have access to location coordinates of the destination location, and may determine when the vehicle has arrived at the location coordinates of the destination location.

At block 404, the device may generate directions from the vehicle (e.g., at the destination location) to a physical structure (e.g., the physical location 118 of FIG. 1) at the destination location. For example, the destination location may include a parking lot or a street, and the physical location may be adjacent to the vehicle at the destination, or may require some walking or other transport of the passenger to the physical location (e.g., from a parking lot or street to a building). In this manner, the directions may be used to help the passenger arrive at the actual location they are intended to be (e.g., the building, residence, etc.) rather than the general location such as a parking lot or street outside of the physical structure. At block 406, the device may present the directions or send the directions to another device for presentation to the passenger.

At block 408, the device may detect an image of the passenger outside of the vehicle at the destination location. The vehicle may capture one or more images (e.g., the images 111 of FIG. 1) of the passenger once the passenger has exited the vehicle, allowing the device to perform image analysis techniques (e.g., object and/or facial recognition, computer vision, etc.) at block 410 to identify whether the passenger has or does not have any objects they are supposed to have or not supposed to have (e.g., as provide to the device by user input), whether the user's facial expression indicates confusion or frustration, whether the user's gait is indicative of confusion or frustration, and/or other user information of the passenger as they are leaving the vehicle.

At block 412, based on the user information identified from the image data and based on device location data of a device of the passenger (e.g., the device 112 of FIG. 1), the device may detect a disorientation event of the passenger. Alternatively or in addition, a disorientation event may be indicated by a user input that provides information regarding whether the user has a condition that may result in a disorientation event (e.g., Alzheimer's, Autism, or the like, with user consent and in accordance with relevant laws).

In some embodiments, with reference to block 412, a disorientation event of a person may include memory loss, inability to navigate to a location, being at a location for longer than an expected time, being stationary or within a small area for longer than a threshold time, moving at a speed lower than a threshold, being outside of a specified location/boundary (e.g., based on geographical coordinates, geo-fencing, etc.), having vital signs that are above or below respective thresholds (e.g., indicative of fatigue, stress, etc.), taking too long to complete a task or set of tasks, and the like.

In some embodiments, with reference to block 412, detection of a disorientation event may use a combination of image data, device location data, device motion data, and/or biometric sensor data. For example, with user consent and in accordance with relevant laws, a user's location may be monitored using device location data (e.g., global navigation satellite system data, Wi-Fi data, Bluetooth data, etc.), a user's state of being may be monitored using images and/or sensor data (e.g., images used for analysis to detect facial expressions, items in a person's possession, attire, gait, injuries, and the like, and biometric sensor data such as body temperature, heartrate, breathing rate, pulse, etc., such as provided by the one or more devices 206 of FIG. 2), and a user's activity may be monitored using device motion data (e.g., accelerometer data indicative of a person falling down or moving in a manner that is unexpected or indicative of stress).

At block 414, the device may generate, based on the detection of the disorientation event, instructions to present to the passenger (and/or to another user as shown in FIG. 1). The instructions may include maps and directions to the physical structure (e.g., from the vehicle) and/or to interior locations of the physical structure, reasons for the passenger being at the location (e.g., inputted or learned), tasks for the passenger to complete at the physical location, items to purchase that physical location, expected time durations for the passenger to be at a particular location and/or to perform a task, indications of how much time of the expected time is remaining or whether the expected time has expired, queries asking the passenger to confirm their status, suggestions to eat, drink, take a break, use the restroom, and the like. At block 416, the device may send the instructions to one or more devices for presentation.

The examples presented herein are not meant to be limiting.

FIG. 5 is a block diagram illustrating an example of a computing device or computer system upon which any of one or more techniques (e.g., methods) may be performed, in accordance with one or more example embodiments of the present disclosure.

For example, the computing system 500 of FIG. 5 may include or represent at least some components of the vehicle 104 of FIG. 1, the vehicle 202 of FIG. 2, the remote system 204 of FIG. 2, the one or more devices 206 of FIG. 2, and/or the vehicle 301 of FIG. 3, and therefore may allow for the selection of activities, the requesting of codes corresponding to the activities, the receiving of codes, and the presentation of codes. The computer system (system) includes one or more processors 502-506. Processors 502-506 may include one or more internal levels of cache (not shown) and a bus controller (e.g., bus controller 522) or bus interface (e.g., I/O interface 520) unit to direct interaction with the processor bus 512.

Processor bus 512, also known as the host bus or the front side bus, may be used to couple the processors 502-506, and a passenger assist device 519 (e.g., for facilitating any of the functions described with respect to FIGS. 1-4) with the system interface 524.

System interface 524 may be connected to the processor bus 512 to interface other components of the system 500 with the processor bus 512. For example, system interface 524 may include a memory controller 518 for interfacing a main memory 516 with the processor bus 512. The main memory 516 typically includes one or more memory cards and a control circuit (not shown). System interface 524 may also include an input/output (I/O) interface 520 to interface one or more I/O bridges 525 or I/O devices 530 with the processor bus 512. One or more I/O controllers and/or I/O devices may be connected with the I/O bus 526, such as I/O controller 528 and I/O device 530, as illustrated.

I/O device 530 may also include an input device (not shown), such as an alphanumeric input device, including alphanumeric and other keys for communicating information and/or command selections to the processors 502-506, and/or the passenger assist device 519. Another type of user input device includes cursor control, such as a mouse, a trackball, or cursor direction keys for communicating direction information and command selections to the processors 502-506, and for controlling cursor movement on the display device.

System 500 may include a dynamic storage device, referred to as main memory 516, or a random access memory (RAM) or other computer-readable devices coupled to the processor bus 512 for storing information and instructions to be executed by the processors 502-506 and/or the passenger assist device 519. Main memory 516 also may be used for storing temporary variables or other intermediate information during execution of instructions by the processors 502-506 and/or the passenger assist device 519. System 500 may include read-only memory (ROM) and/or other static storage device coupled to the processor bus 512 for storing static information and instructions for the processors 502-506 and/or the passenger assist device 519. The system outlined in FIG. 5 is but one possible example of a computer system that may employ or be configured in accordance with aspects of the present disclosure.

According to one embodiment, the above techniques may be performed by computer system 500 in response to processor 504 executing one or more sequences of one or more instructions contained in main memory 516. These instructions may be read into main memory 516 from another machine-readable medium, such as a storage device. Execution of the sequences of instructions contained in main memory 516 may cause processors 502-506 and/or the passenger assist device 519 to perform the process steps described herein. In alternative embodiments, circuitry may be used in place of or in combination with the software instructions. Thus, embodiments of the present disclosure may include both hardware and software components.

According to one embodiment, the processors 502-506 may represent machine learning models. For example, the processors 502-506 may allow for neural networking and/or other machine learning techniques used to operate the vehicle 202, the remote system 204, and/or the one or more devices 206 of FIG. 2.

In one or more embodiments, the computer system 500 may perform any of the steps of the processes described with respect to FIG. 4.

In one or more embodiments, the computer system 500 may include image devices 532 (e.g., cameras, such as to capture the images 111 of FIG. 1).

In one or more embodiments, the computer system 500 may include an HMI 534 (e.g., corresponding to the infotainment system 302 of FIG. 3) with which to present directions/maps and/or other instructions (e.g., such as those presented using the device 112 of FIG. 1).

Various embodiments may be implemented fully or partially in software and/or firmware. This software and/or firmware may take the form of instructions contained in or on a non-transitory computer-readable storage medium. Those instructions may then be read and executed by one or more processors to enable the performance of the operations described herein. The instructions may be in any suitable form, such as, but not limited to, source code, compiled code, interpreted code, executable code, static code, dynamic code, and the like. Such a computer-readable medium may include any tangible non-transitory medium for storing information in a form readable by one or more computers, such as but not limited to read-only memory (ROM); random access memory (RAM); magnetic disk storage media; optical storage media; a flash memory, etc.

A machine-readable medium includes any mechanism for storing or transmitting information in a form (e.g., software, processing application) readable by a machine (e.g., a computer). Such media may take the form of, but is not limited to, non-volatile media and volatile media and may include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, solid state devices (SSDs), and the like. The one or more memory devices (not shown) may include volatile memory (e.g., dynamic random access memory (DRAM), static random access memory (SRAM), etc.) and/or non-volatile memory (e.g., read-only memory (ROM), flash memory, etc.).

Computer program products containing mechanisms to effectuate the systems and methods in accordance with the presently described technology may reside in main memory 516, which may be referred to as machine-readable media. It will be appreciated that machine-readable media may include any tangible non-transitory medium that is capable of storing or encoding instructions to perform any one or more of the operations of the present disclosure for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures.

Embodiments of the present disclosure include various steps, which are described in this specification. The steps may be performed by hardware components or may be embodied in machine-executable instructions, which may be used to cause a general-purpose or special-purpose processor programmed with the instructions to perform the steps. Alternatively, the steps may be performed by a combination of hardware, software, and/or firmware.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations together with all equivalents thereof.

The operations and processes described and shown above may be carried out or performed in any suitable order as desired in various implementations. Additionally, in certain implementations, at least a portion of the operations may be carried out in parallel. Furthermore, in certain implementations, less than or more than the operations described may be performed.

The word “exemplary” is used herein to mean “serving as an example, instance, or illustration.” Any embodiment described herein as “exemplary” is not necessarily to be construed as preferred or advantageous over other embodiments.

As used herein, unless otherwise specified, the use of the ordinal adjectives “first,” “second,” “third,” etc., to describe a common object, merely indicates that different instances of like objects are being referred to and are not intended to imply that the objects so described must be in a given sequence, either temporally, spatially, in ranking, or any other manner.

It is understood that the above descriptions are for purposes of illustration and are not meant to be limiting.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

What is claimed is:

1. A method for assisting a vehicle passenger who has exited the vehicle, the method comprising:
detecting, by at least one processor of a first device, based on first location data associated with a vehicle, that the vehicle has arrived at a destination location for a passenger of the vehicle;
generating, by the at least one processor, directions from the vehicle at the destination location to a physical structure at the destination location;
causing presentation, by the at least one processor, of the directions;
detecting, by the at least one processor, an image of the passenger exterior to the vehicle at the destination location;
identifying, by the at least one processor, based on the image, user information associated with the passenger at the destination location;
detecting, by the at least one processor, at a time when the passenger is exterior to the vehicle and after the vehicle has arrived at the destination location, based on the user information and second location data associated with a second device of the passenger, a disorientation event associated with the passenger;
generating, by the at least one processor, based on the detecting of the disorientation event, instructions to be presented to the passenger; and
causing presentation, by the at least one processor, of the instructions at the second device.

2. The method of claim 1, wherein the directions are first directions from a first location of the vehicle to the physical structure, the method further comprising:
generating second instructions to be presented to the passenger, the second instructions comprising a map and second directions from the physical structure to a second location of the vehicle; and
causing presentation of the second instructions at the second device.

3. The method of claim 1, wherein the instructions comprise a map of locations inside of the physical structure, and wherein the instructions further comprise directions from the vehicle to a location of the locations inside of the physical structure.

4. The method of claim 1, wherein the instructions comprise a list of items for the passenger to possess or purchase.

5. The method of claim 1, wherein the instructions comprise a reminder of a task for the passenger to complete at the destination location and an expected time duration for the passenger to complete the task.

6. The method of claim 1, wherein the instructions comprise directions from the physical structure to the vehicle.

7. The method of claim 1, further comprising:
causing presentation of second instructions at a third device associated with a person different than the passenger, wherein the second instructions are indicative of the detecting of the disorientation event.

8. The method of claim 1, further comprising:
detecting biometric sensor data associated with the passenger,
wherein detecting the disorientation event is further based on the biometric sensor data.

9. The method of claim 1, wherein the user information comprises at least one of an item possessed by the passenger in the image, a gait of the passenger in the image, or a facial expression of the passenger in the image.

10. The method of claim 1, further comprising:
generating a geo-fence associated with the physical structure; and
detecting, based on the second location data, that the passenger is exterior to the geo-fence,
wherein detecting the disorientation event is further based on detecting that the passenger is exterior to the geo-fence.

11. The method of claim 1, further comprising:
detecting, based on the second location data, that an amount of time spent by the passenger at the physical structure exceeds a threshold time,
wherein detecting the disorientation event is further based on detecting that the amount of time exceeds the threshold time.

12. The method of claim 1, further comprising:
detecting, based on device motion data associated with the second device, that a speed at which the passenger is moving is below a threshold speed,
wherein detecting the disorientation event is further based on detecting that the speed is below the threshold speed.

13. The method of claim 1, further comprising:
generating second instructions to be presented to the passenger using a human machine interface of the vehicle; and
causing presentation of the second instructions using the human machine interface,
wherein the second instructions comprise at least one of a task for the passenger to complete at the destination location or a time duration for the passenger to be at the destination location.

14. Computer-readable storage hardware storing information configured to, when executed by a computing device, cause the computing device to perform a process, the process comprising:
detecting, based on first location data associated with a vehicle, that the vehicle has arrived at a destination location for a passenger of the vehicle;
generating directions from the vehicle at the destination location to a physical structure at the destination location;
detecting an image of the passenger exterior to the vehicle at the destination location;
identifying, based on the image, user information associated with the passenger at the destination location;
detecting, at a time when the passenger is exterior to the vehicle and after the vehicle has arrived at the destination location, based on the user information and second location data associated with a device of the passenger, a disorientation event associated with the passenger;
generating, based on the detecting of the disorientation event, instructions to be presented to the passenger; and
causing presentation of the instructions at the device.

15. The computer-readable storage hardware of claim 14, wherein the directions are first directions from a first location of the vehicle to the physical structure, the process further comprising:
generating second instructions to be presented to the passenger, the second instructions comprising a second map and second directions from the physical structure to a second location of the vehicle; and
causing presentation of the second instructions at the device.

16. The computer-readable storage hardware of claim 14, wherein the instructions comprise a map of locations inside of the physical structure, and wherein the instructions further comprise directions from the vehicle to a location of the locations inside of the physical structure.

17. The computer-readable storage hardware of claim 14, wherein the instructions comprise a reminder of a task for the passenger to complete at the destination location and an expected time duration for the passenger to complete the task.

18. The computer-readable storage hardware of claim 14, the process further comprising:
detecting biometric sensor data associated with the passenger,
wherein detecting the disorientation event is further based on the biometric sensor data.

19. The computer-readable storage hardware of claim 14, wherein the user information comprises at least one of an item possessed by the passenger in the image, a gait of the passenger in the image, or a facial expression of the passenger in the image.

20. A device for assisting a vehicle passenger who has exited the vehicle, the device comprising at least one processor coupled to memory, the at least one processor configured to:
detect, based on first location data associated with a vehicle, that the vehicle has arrived at a destination location for a passenger of the vehicle;
generate directions from the vehicle at the destination location to a physical structure at the destination location;
cause presentation of the directions;
detect an image of the passenger exterior to the vehicle at the destination location;
identify, based on the image, user information associated with the passenger at the destination location;
detect, at a time when the passenger is exterior to the vehicle and after the vehicle has arrived at the destination location, based on the user information and second location data associated with a second device of the passenger, a disorientation event associated with the passenger;
generate, based on the detecting of the disorientation event, instructions to be presented to the passenger; and
cause presentation of the instructions at the second device.
